(19) United States
(12) Patent Application Publication
(10) Pub. No.: US 2007/0129798 A1
(43) Pub. Date: Jun. 7, 2007

(54) Intraocular device

(76) Inventor: Satish Chawdhary, Staffordshire (GB)

Correspondence Address: SMITH-HILL AND BEDELL, P.C., 16100 NW CORNELL ROAD, SUITE 220, BEAVERTON, OR, 97006, US

(21) Appl. No.: 10/568,553

(22) PCT Filed: Jul. 19, 2004
(86) PCT No.: PCT/GB04/03110

§ 371 Date: Feb. 17, 2006

(30) Foreign Application Priority Data
Aug. 19, 2003 (GB) 0319408.1

(51) Int. Cl. A61F 2/16

(52) U.S. Cl. 623/6.13; 623/6.36; 623/6.34; 623/6.37

(57) ABSTRACT

An intraocular device for implanting within a patient's eye for enabling the eye to alter its focusing power includes an optic member, preferably in the form of an optic, which is designed to locate in the patient's eye in the line of sight such that the patient sees through the optic. The device further includes means for altering the shape of the optic to alter its focusing power and thereby alter the focus of the patient's eye. An implant for insertion into a patient's eye is also disclosed, the implant including an artificial lens to which the intraocular device is preferably attached, the artificial lens and the optic member of the intraocular device being positioned in the line of sight such that the patient sees through both.

The present application relates to an intraocular device for implanting within a patient's eye, for enabling the eye to alter its focussing power.

A normal eye includes a lens which in its relaxed condition allows for clear vision at a distance. For near vision, for example for reading fine print or threading a needle, the focussing power of the eye has to be increased. This increase in focussing power is achieved through the contraction of the ciliary muscle in the eye, which effectively deforms the natural lens, making it more convex and increasing its focussing power. This process is called accommodation, and allows the eye to selectively focus for objects at various distances.

Every person in about their mid forties loses the capability of accommodation, due to the increasing rigidity of the natural lens of the eye and, to some extent, by the weakening power of the ciliary muscle. This leads to the condition called presbyopia. Thus presbyopia is a condition in which despite having normal distance vision, with or without glasses, the affected person is unable to see with clarity at near distances, for example when reading a book. Once presbyopia sets in, reading spectacles are needed to read fine print, even by those who do not need to use glasses for distance vision. Every person eventually suffers from presbyopia, and indeed aging over 45 years is the commonest cause of presbyopia.

Every person also becomes absolutely presbyopic after cataract surgery. In such surgery, the natural lens is replaced with an implant in the form of an artificial lens. The artificial lenses currently used to replace the natural lens are made of rigid plastic and have only one fixed power, mainly for seeing well for distance. Therefore the eye cannot increase its power for near vision, even though the ciliary muscle can contract normally. So, people after cataract surgery have to rely on reading glasses, even when their distance vision is good without glasses.

Thus everyone has to rely on reading glasses after a certain age (typically mid forties) and after cataract surgery.

There are currently artificial lenses available for use during cataract surgery which attempt to solve the above problem. One artificial lens focuses light at multiple different points inside the eye. Most of the light is focussed for distance vision, whilst some is focussed for near vision and intermediate vision. This relies on the brain effectively deciding which light to concentrate on. It can reduce contrast of vision, cause halos around lights and can cause double vision. Another similar artificial lens has three zones with different focussing powers, providing for three different focal distances within the eye as the lens splits the light into three bundles. This also suffers from the disadvantages of reduced contrast, double vision and halos around the light.

Another prior art artificial lens is able to move forward slightly through contraction of muscles within the eye. However, the lenses can only move forward by 0.75 mm at the most, whereas for good accommodation up to 2.5 mm of forward movement would be needed. Thus, these artificial lenses are not widely used at present.

According to the invention there is provided an intraocular device for implanting within a patient's eye, the device including an optic member for location in the eye such that the patient sees through the optic member, and means for altering the shape of the optic member to alter its focussing power and thereby alter the focus of the patient's eye.

The optic member may be configured for location in front of the normal lens of the eye. The normal lens of the eye may be the eye's natural lens or an artificial implanted lens. Preferably, the optic member is configured for location outside the capsular bag which contains the normal lens of the eye.

The shape of the optic member may be alterable between a relaxed shape in which it provides substantially no focussing effect and a focussing shape in which it provides between 3 and 6, and preferably between 4 and 5dioptres of focussing power.

In the focussing shape, the optic member is preferably convex on at least one side.

The optic member may be caused to alter its shape from the relaxed to the focussing shape in response a stimulus which causes focussing of the lens of a normal young eye. This stimulus may comprise or cause the contraction of the ciliary muscle of the eye. The extent of change in shape and focussing effect of the optic member is preferably a function of (and may be proportional to) the magnitude of the stimulus, for example the extent of the contraction of the ciliary muscle.

The intraocular device may include a fluid reservoir. The optic member may include a central cavity which is in communication with the fluid reservoir. The reservoir may contain a volume of fluid, which is preferably a transparent liquid. The means for altering the shape of the optic member may include means for causing fluid to move from the reservoir into or out of the central cavity of the optic member.

Preferably the device is configured such that fluid moves into the cavity in response to contraction of the ciliary muscle. Preferably the amount of fluid moving into the cavity is a function of the extent of contraction of the ciliary muscle and may be proportional thereto.

Preferably the cavity is defined within walls which are biased into a position in which the cavity contains substantially no fluid, but which may flex into a position in which the cavity contains fluid. Preferably fluid is caused to move from the cavity into the reservoir in response to relaxation of the ciliary muscle, allowing the bias of the walls of the cavity to force the fluid back into the reservoir.

The walls of the central cavity may include an anterior wall and a posterior wall between which the central cavity is defined. The anterior wall preferably comprises a flexible, substantially transparent membrane which in an unstressed condition is substantially planar. The posterior wall may comprise a planar, transparent member, which may be rigid. When there is substantially no fluid in the cavity, the anterior and posterior members may lie substantially adjacent to one another, causing the central cavity to have substantially no volume.

When fluid is present in the reservoir, the anterior wall preferably flexes away from the posterior wall, into a convex shape.

The anterior wall preferably has a sufficient degree of elasticity and elastic memory that it returns to its unstressed, planar condition on relaxation of the ciliary muscle.

The intraocular device may further include a conduit which provides a fluid connection between the central cavity in the optic member and the reservoir. The conduit may comprise a capillary tube connecting the reservoir and the cavity. The capillary tube may have an internal diameter of between 0.5 and 1.5 mm and may be transparent. The tube is preferably substantially rigid so that it does not deform when fluid is conveyed through it.

The reservoir is preferably configured for location adjacent to ciliary muscle in the ciliary sulcus of the patient's eye.

Preferably the reservoir is shaped such that when it is in place adjacent the ciliary muscle, contraction of the ciliary muscle causes the compression of the reservoir, thus forcing fluid to the cavity in the optic member.

The reservoir preferably includes a peripheral part which abuts against the ciliary muscle, the peripheral part being flexible. The reservoir preferably further includes a base part, the peripheral part and base part together defining a chamber for the fluid. The base part is preferably substantially rigid.

Preferably the reservoir is configured such that contraction of the ciliary muscle causes compression of the peripheral part, thus forcing fluid from the reservoir into the central cavity of the optic member.

The device may include two reservoirs which may be configured to diametrically opposed, for example in top and bottom regions of the ciliary sulcus of eye, each reservoir being connected to the optic member via a capillary tube.

The optic member may alternatively be configured for location in the region of the eye to the rear of the iris. The optic member may be spaced from the normal lens of the eye. Alternatively the optic member may locate adjacent to the normal lens.

The optic member may be configured to locate in front of the iris of the eye.

According to the invention there is further provided an implant for insertion into a patient's eye, the implant including an artificial lens and an intraocular device according to any of the preceding nineteen paragraphs, the artificial lens and the optic member of the intraocular device being positioned in the line of sight such that the patient sees through both.

Preferably, the artificial lens is substantially rigid.

Preferably the optic member is positioned adjacent to the artificial lens. The optic member may cover a central part of the anterior surface of the artificial lens. The optic member may be fused with the central part of the artificial lens.

Preferably the intraocular device is configured for location in the ciliary sulcus of the eye, between the lens and the iris. Preferably the intraocular device is configured for location outside the capsular bag, behind the iris, in contact with the ciliary muscle, in a position to be compressed by the contracting ciliary muscle.

Embodiments of the invention will be described for the purpose of illustration only with reference the accompanying drawings in which:

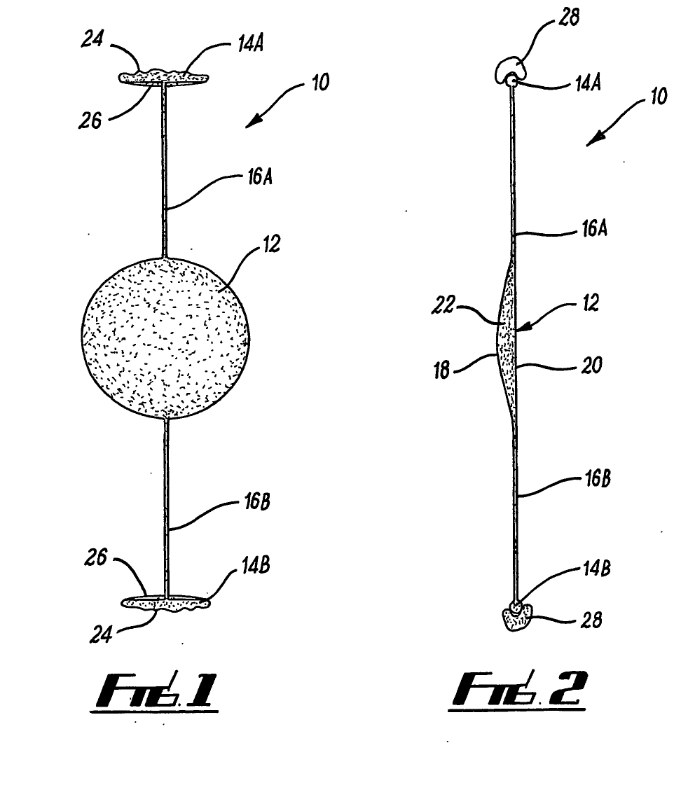

FIG. 1 is a front view of an intraocular device according to the invention;

FIG. 2 is a side view of the device of FIG. 1;

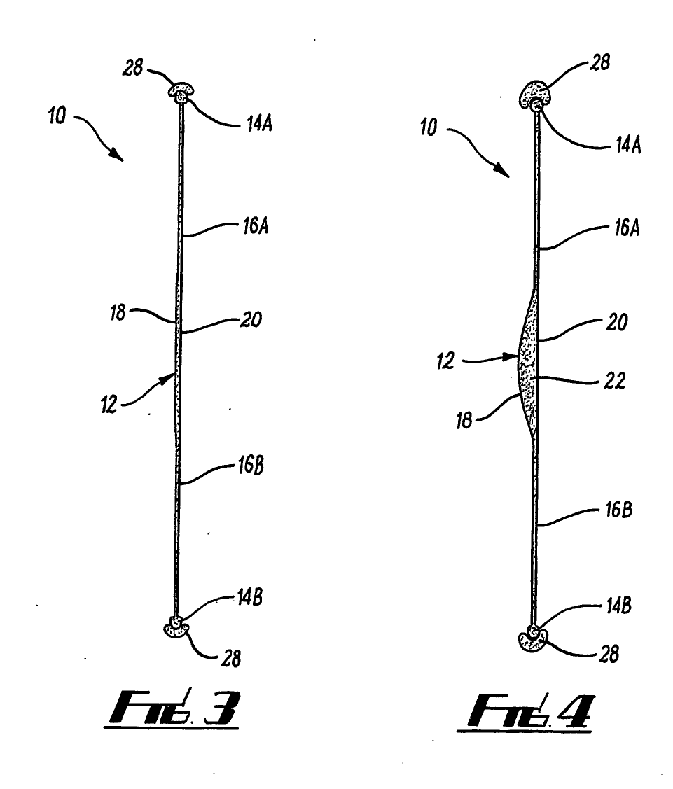

FIG. 3 is a side view of the device of FIG. 1 in a relaxed state;

FIG. 4 is a side view of the device of FIG. 1 in a focussing state;

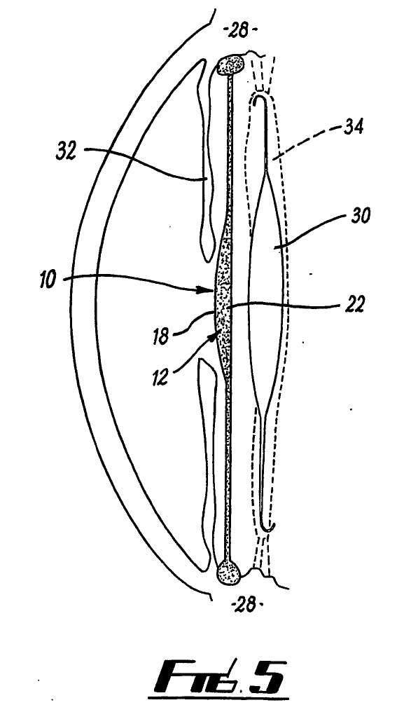

FIG. 5 is a sectional view illustrating the device of FIGS. 1 to 4 in place in a patient's eye, with a standard rigid lens implanted in the capsular bag after a cataract operation;

FIG. 6 is a diagrammatic front view of the device according to the invention fused to a rigid artificial lens, and located in an alternative position in a patient's eye;

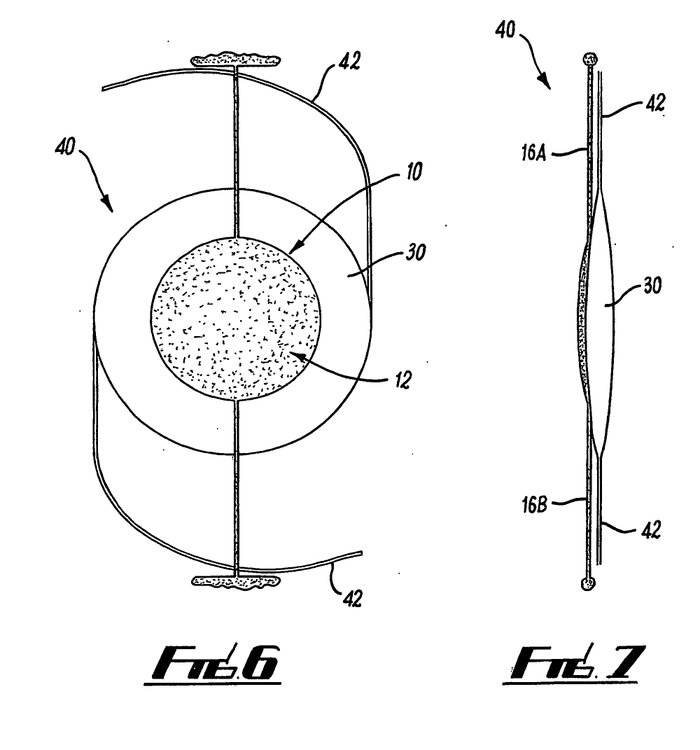

FIG. 7 is a side view of the device of FIG. 6;

FIG. 8 is a sectional view of the device of FIGS. 6 and 7 in place in a patient's eye;

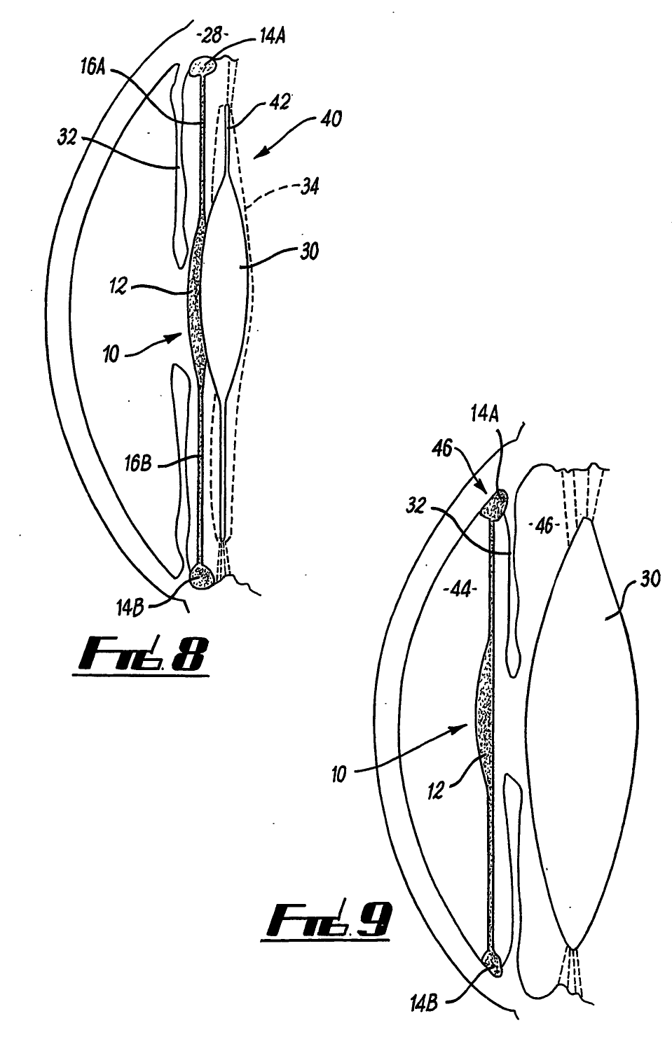

FIG. 9 is a sectional view of the device according to the invention in an alternative location in a patient's eye;

FIG. 10 is a diagrammatic partial side view illustrating an alternative embodiment of the invention;

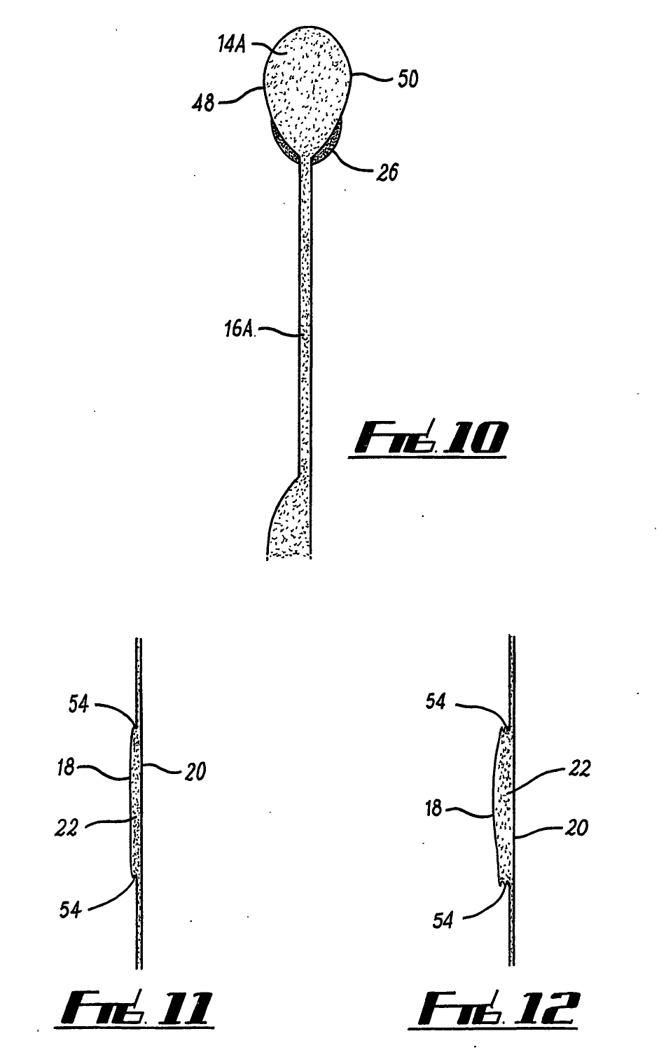

FIG. 11 is a diagrammatic sectional view of an alternative embodiment of the invention; and

FIG. 12 is a diagrammatic side view of the embodiment of FIG. 11 in an alternative condition.

Referring to FIGS. 1 to 4, there is illustrated an intraocular device 10 for implanting within a patient's eye. The intraocular device 10 is for use in improving a patient's vision when they have become presbyopic, for example after cataract surgery or simply due to age.

The intraocular device 10 includes an optic member in the form of an optic 12 which is designed to locate in the patient's eye in the line of sight such that the patient sees through the optic. The intraocular device 10 is designed such that the shape of the optic 12 may be altered in order to alter its focussing power and thereby alter the focus of the patient's eye, as described in more detail hereinafter.

The optic 12 preferably locates in front of a patient's own lens (natural lens or rigid lens implant), outside the capsular bag which contains the lens (this is referred to as a “piggy back” device).

Referring to FIG. 1, the optic 12 is generally circular in plan view and about 4 mm in diameter (but could typically be between 4 mm and 6 mm).

The intraocular device 10 further includes an upper reservoir 14A and a lower reservoir 14B. The reservoirs are filled with a fluid which is transparent but which has a high refractive index.

The upper reservoir 14A is connected to an upper circumferential part of the optic 12 by a capillary tube 16A and the lower reservoir 14B is connected to a lower peripheral part of the optic 12 by a capillary tube 16B.

The diameter of the optic 12 is about 4 to 6 mm, with the overall end to end measurement of the device (from the reservoir 14A to the reservoir 14B) being about 12 to 14 mm, to fit the dimensions of the ciliary sulcus of the eye.

Referring in particular to FIGS. 2 to 4, the optic 12 includes an anterior wall 18 and a posterior wall 20. A cavity 22 is defined between the walls 18 and 20. The volume of the cavity 22 can vary from substantially zero as in FIG. 3 to a volume sufficient to force the anterior wall into a convex shape as in FIGS. 2 and 4, and described in more detail below.

The anterior, wall 18 of the optic 12 comprises a flexible, transparent membrane with rubbery characteristics, i.e. it has a high elastic memory and tensile strength. The posterior wall 20 is substantially rigid and transparent, normally having a basal power of zero (although it could be, by choice a low minus or low plus value). This is so that the wall itself does not have a significant focussing effect.

The cavity 22 is connected to the upper reservoir 14A via the capillary tube 16A, and to the lower reservoir 14B via the capillary tube 16B. This means that fluid can flow between the reservoirs 14A, 14B and the cavity 22.

Each reservoir 14A, 14B includes' a soft peripheral part 24 made of a silicone membrane or other flexible material and a substantially rigid base part 26 which may be made of polymethylmethacrylate (PMMA) or a similar rigid material. The peripheral part 24 of the reservoir 14A, B rests in the ciliary sulcus of the eye, abutting against an anterior strip of ciliary muscle 28. This is the muscle which contracts when the eye focuses for near vision.

FIG. 5 illustrates the positioning in the eye of the intraocular device 10 according to one embodiment of the invention. The device 10 is located outside the capsular bag 34 of the eye, with the reservoirs 14a and 14b in the ciliary sulcus of the eye, between a lens 30 and iris 32 of the eye. FIG. 5 illustrates an eye which has undergone cataract surgery so that the natural lens of the eye has been replaced with a rigid monofocal lens 30 which is located in the capsular bag 34 of the eye. The functioning of the intraocular device 10 will initially be described with reference to this embodiment.

When the eye is being used for distance vision, the intraocular device 10 is in the condition illustrated in FIG. 3. The ciliary muscle 28 is relaxed and each reservoir 14A, 14B is relatively full of fluid. There is therefore nothing to force the anterior wall 18 of the optic 12 out of its relaxed position, which is generally planar. The intraocular device 10 has no effect on the focussing power of the lens .30 which therefore allows the eye to focus for distance vision.

When the eye focuses for near vision, the ciliary muscle 28 contracts. This contraction squeezes the peripheral part 24 of each reservoir 14A, 14B, thus driving the fluid from the reservoir through the capillary tubes 16A, 16B into the cavity 22 of the optic 12. This causes the flexible, elastic anterior wall 18 of the cavity 22. to curve in to a convex ‘balloon’shape, as illustrated in FIGS. 2 and 4. This, increased curvature and increased thickness, together with the high refractive index fluid in the cavity 22 of the optic 12 increases the power of the optic, enabling the eye to focus for near vision.

Once the eye stops focussing for near vision, the ciliary muscle 28 relaxes and the elastic memory of the anterior wall 18 causes it to return to its planar position, collapsing the balloon and returning the power of the optic 12 to its basic resting level. The fluid is pushed back through the capillary tubes 16A, 16B into the reservoirs 14A, 14B. The reservoirs 14A, 14B are able to accommodate the fluid due to the relaxation of the ciliary muscle 28.

The extent of ciliary muscle contraction is proportional to the degree of close focussing required. Therefore, the amount of fluid pushed by the ciliary muscle into the cavity 22 will depend upon the degree of close focussing required, thus ensuring that the thickness and focussing power of the optic 12 is appropriate. The device 10 therefore mirrors the functioning of the healthy, young eye.

To ensure effective functioning of the device, it is desirable that the fluid has a low viscosity, so that it may be moved quickly and predictably in a reversible manner, with a low driving force. It should also have a high refractive index typically between 1.3 and 1.5 in order that the least volume shift is required to achieve the required increase in focussing power of the optic. It should also be colourless and transparent and non toxic, in case it leaks inside the eye. The fluid may be a balance salt solution but other fluids may be suitable.

The anterior wall 18 ideally has a high degree of elasticity so that in the resting state it is substantially planar and the fluid remains in the reservoir 22 and capillary tubes 16A, 16B only. The anterior wall 18 should also have a high degree of elastic memory so that over time it does not lose its initial taughtness. The material should also be transparent, have a high refractive index and have good optical surface characteristics so that it can work as an effective refractive surface when the cavity is both inflated and deflated.

In relation to the reservoir 14A, 14B, the peripheral part 24, which abuts snugly against the ciliary muscle 28, should be of a soft membrane (eg silicone) so the contracting ciliary muscle can easily compress it and also such that it will not erode into the ciliary muscle. The base part 26 of the reservoir should be substantially rigid and may be of a plastics material such as PMMA so that essentially no compressive force is used up in redistribution of the fluid within the reservoir itself but if only used in causing the migration of the fluid into the cavity 22 in the optic 12.

The capillary tubes 16A, 16B may be semi rigid so that they can be bent/flexed to allow insertion of the device into the eye through a small incision. However, the tubes should not be able to stretch under fluid pressure as fluid is conveyed to the central cavity. The capillary tubes may be made of a flexible acrylic material. The tubes are preferably transparent so that they do not affect the quality of the optical part of the lens 30 behind them. They require a narrow internal bore in order that they act as optimal hydraulic systems, connecting much wider cavities i.e. the reservoirs 14 and central cavity 22.

Referring to FIGS. 6 & 7, there is illustrated an alternative embodiment of the invention in which the intraocular device 10 forms part of an implant 40. The implant 40 comprises the intraocular device 10 and an artificial lens 30 to which the intraocular device 10 is attached.

The lens 30 is provided with fixation haptics 42 which help to locate it in the capsular bag 34 of the eye, in the correct position for vision. In this embodiment, the intraocular device 10 is fused to the central 4mm or so of the lens 30.

Referring to FIG. 8, the whole implant 40 is placed within the eye, with the lens 30 and the fixation haptics 42 being inside the capsular bag 34. The intraocular device 10 is located with the reservoirs 14a and 14b resting in the ciliary sulcus region, with the reservoirs 14A, 14B being in contact with the ciliary muscle 28 as described previously. The implant 40 allows an individual being treated for cataracts to maintain or re-gain close vision.

FIG. 9 illustrates the use of an intraocular device 10 in an eye being treated for normal age-induced presbyopia. In this case, the eye retains its natural lens 30 and the intraocular device 10 may be placed in the anterior chamber 44 of the eye 28, in front of the iris 32. The reservoirs 14A, 14B are placed in the angle region 46 against the ciliary muscle band. If this does not provide enough power from the ciliary muscle contraction, the reservoirs 14 can be placed through peripheral iridectomies (i.e. surgically made holes in the iris) 180° apart, into the ciliary sulcus, against the ciliary muscle. Alternatively, the intraocular device 10 could be placed entirely in the posterior chamber 46 (i.e. behind the iris 32) with the optic 12 resting on the lens 30, provided that the optic 12 is made of a biocompatible material and does not produce cataract.

With advancing age, the ciliary muscle on which the intraocular device 10 depends may become too weak to generate enough power to drive the fluid into the cavity 22, leading to failure of the device. In order to overcome this problem, which will tend to occur with very elderly patients, an external source of power may be incorporated in order to achieve movement of the fluid.

Referring to FIG. 10, the source of power could be a magnetic field, which may be used to compress the reservoir 14A, 14B to push the fluid towards the cavity 22. A posterior wall 50 of the reservoir may be internally coated with a ferro magnetic substance (which magnets attract) and the anterior wall 48 of the reservoir 14 may be coated with an apomagnetic substance (which magnets repel, i.e. another magnet or bismuth). An externally applied magnetic field will then push the anterior wall 48 of the reservoir back and pull the posterior wall 50 forward, thus achieving compression of the reservoir without any overall movement of the device itself. In this way, the device 10 can respond to external sources of power even once the ciliary muscle has failed in extreme old age.

There is thus provided a device which will help to restore accommodation in eyes that have lost this ability after cataract lens implant surgery. The device may also be used to restore accommodation in presbyopia caused by the normal aging process.

The product may therefore be used to avoid the need for reading glasses in:
1. Patients who. have had cataract surgery in the past with a rigid lens implant and therefore have lost accommodation.
2. Patients who are undergoing cataract surgery, so that they do not lose accommodation and become absolutely presbyopic.
3. Patients with age onset presbyopia and refractive errors, who can be provided with a replacement clear lens without losing accommodation.
4. Patients with age onset presbyopia, who do not wish their natural lens to be replaced with an artificial lens but wish to restore their ability for accommodation.

Therefore, the intraocular device according to the invention is able to assist all persons more than about forty-five years of age and anyone whose natural lens is removed surgically for any reason, such as cataract surgery or because of a lens injury or a refractive error correction.

The intraocular device works on principles which closely resemble naturally occurring accommodation, in contrast to other products in this field.

The device changes the power of the lens in a way which is equivalent to the normal process of accommodation in the healthy young eye and does not depend on the principle of splitting light into different focal distances or shifting focus by a forward movement of the lens.

The above described intraocular device is particularly advantageous in that the device (and in particular the reservoirs 14a , 14b ) are located outside the capsular bag 34 of the eye.

The contraction of the ciliary muscle in a young eye causes the lens to deform to a near focus state only because the normal (surgically untouched) capsular bag remains elastic. The zonules which attach the capsular bag to the ciliary muscle successfully transmit the effect of ciliary muscle contraction to the jelly-like consistency of the natural lens. However, once the original lens has been scooped out, as with cataract surgery, the capsular bag is scarred and within weeks it loses its elasticity and becomes rigid. Thus, the effect of the contraction of the ciliary muscle ceases to reach the lens. The fibrosing forces of the scarred capsular bag are so great that the capsular bag clasps around the implant within weeks of surgery.

The present invention places the intraocular device outside the capsular bag, thus avoiding the compressive forces of the shrunken capsule, which would otherwise not allow the optic to inflate. According to the above described embodiment, the reservoirs 14a and 14b are snugly in contact with the ciliary muscle. Thus, the shrunken and ineffective transmission mechanism caused by the scarred capsular bag is bypassed. The ciliary muscle is not acting through any intermediaries and is compressing and squeezing the reservoirs 14a and 14b directly.

Various modifications may be made to the above described embodiments without departing from the scope of the invention. For example, referring to FIGS. 11 and 12, the anterior wall 18 may be minimally convex and made of a thin plate of transparent, rigid plastics material, with perfect optical characteristics. The anterior wall 18 may be attached (like an apical cap) to the rigid posterior wall 20 via an elastic membrane 54, which can be spring biased and has micro-corrugations, like the folds in an airbag of an accordion. A cavity 22 would be defined between the anterior and posterior walls and contraction of the ciliary would squeeze fluid into the cavity 22, thus forcing the anterior wall 18 away from the posterior wall 20 in a similar manner to that described above.

In this embodiment of the invention, the design helps to maintain the perfection of the optics of the anterior surface of the anterior wall of the device in its inflated as well as its deflated position. The optical quality of the anterior surface will also not be at risk of degradation over time. The resistance and the rigidity of the corrugations of the elastic membrane 54 may be adjusted and possibly spring biased to ensure the optic is not locked in an inflated or accommodated state (or near focus stage) and to determine how much of an elevation of the anterior wall 18 will occur when the fluid is driven into the cavity 22. The powers of the device will increase by the entry of high refractive index fluid into the cavity 22 as well as by the increase in the thickness of the optical part. The curvature of the anterior wall 18 of the optic 12 will not change in this embodiment of the invention.

In a further modification, the capillary tubes 16a and 16b will not be straight but instead may be curved into a “C” shape or a “J” shape. In addition, the design of and number of reservoirs may be modified. For example, there could be a single reservoir running all around the circumference of the device, having around a 13 to 14 mm diameter, with the optic 12 only occupying the central 4 to 5 mm. The capillary tubes could then be shaped like the spokes of a wheel. In addition, the precise materials used could be altered.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1-51. (canceled)

52. An intraocular device for implanting within a patient's eye, the device including an optic member for location in the eye such that the patient sees through the optic member, and means for altering the shape of the optic member to alter its focussing power and thereby alter the focus of the patient's eye.

53. An intraocular device according to claim 52, wherein the optic member is configured for location in front of the normal lens of the eye.

54. An intraocular device according to claim 52, wherein the optic member is configured for location outside the capsular bag which contains the normal lens of the eye.

55. An intraocular device according to claim 52, wherein the shape of the optic member is alterable between a relaxed shape in which it provides substantially no focussing effect and a focussing shape in which it provides between 3 and 6 dioptres of focussing power.

56. An intraocular device according to claim 55, wherein in the focussing shape, the optic member is convex on at least one side.

57. An intraocular device according to claim 55, wherein the optic member is caused to alter its shape from the relaxed to the focussing shape in response a stimulus which causes focussing of the lens of a normal young eye.

58. An intraocular device according to claim 57, wherein the extent of change in shape and focussing effect of the optic member is a function of the magnitude of the stimulus, for example the extent of the contraction of the ciliary muscle.

59. An intraocular device according to claim 52, wherein the intraocular device includes a fluid reservoir.

60. An intraocular device according to claim 59, wherein the optic member includes a central cavity which is in communication with the fluid reservoir.

61. An intraocular device according to claim 60, wherein the means for altering the shape of the optic member includes means for causing fluid to move from the reservoir into or out of the central cavity of the optic member.

62. An intraocular device according to claim 61, wherein the device is configured such that fluid moves into the cavity in response to contraction of the ciliary muscle.

63. An intraocular device according to claim 62, wherein the amount of fluid moving into the cavity is a function of the extent of contraction of the ciliary muscle and may be proportional thereto.

64. An intraocular device according to claim 63, wherein the cavity is defined within walls which are biased into a position in which the cavity contains substantially no fluid, but which may flex into a position in which the cavity contains fluid, and wherein fluid is caused to move from the cavity into the reservoir in response to relaxation of the ciliary muscle, allowing the bias of the walls of the cavity to force the fluid back into the reservoir.

65. An intraocular device according to claim 64, wherein the walls of the central cavity includes an anterior wall and a posterior wall between which the central cavity is defined, the anterior wall comprising a flexible, substantially transparent membrane which in an unstressed condition is substantially planar, and the posterior wall comprising a planar, transparent rigid member.

66. An intraocular device according to claim 65, wherein when there is substantially no fluid in the cavity, the anterior and posterior members lie substantially adjacent to one another, causing the central cavity to have substantially no volume.

67. An intraocular device according to claim 65, wherein when fluid is present in the reservoir, the anterior wall flexes away from the posterior wall, into a convex shape and wherein the anterior wall has a sufficient degree of elasticity and elastic memory that it returns to its unstressed, planar condition on relaxation of the ciliary muscle.

68. An intraocular device according to claim 60, wherein the intraocular device further includes a conduit which provides a fluid connection between the central cavity in the optic member and the reservoir.

69. An intraocular device according to claim 68, wherein the conduit comprises a capillary tube connecting the reservoir and the cavity.

70. An intraocular device according to claim 59, wherein the reservoir is configured for location adjacent to ciliary muscle in the ciliary sulcus of the patient's eye.

71. An intraocular device according to claim 70, wherein the reservoir is shaped such that when it is in place adjacent the ciliary muscle, contraction of the ciliary muscle causes the compression of the reservoir, thus forcing fluid to the cavity in the optic member.

72. An intraocular device according to claim 70, wherein the reservoir includes a peripheral part which abuts against the ciliary muscle, the peripheral part being flexible, and a base part, the peripheral part and base part together defining a chamber for the fluid, the base part being substantially rigid.

73. An intraocular device according to claim 72, wherein the reservoir is configured such that contraction of the ciliary muscle causes compression of the peripheral part, thus forcing fluid from the reservoir into the central cavity of the optic member.

74. An intraocular device according to claim 73, wherein the device includes two reservoirs which are configured to be diametrically opposed, for example in top and bottom regions of the ciliary sulcus of eye, each reservoir being connected to the optic member via a capillary tube.

75. An intraocular device according to claim 52, wherein the optic member is configured for location in the region of the eye to the rear of the iris.

76. An intraocular device according to claim 52, wherein the optic member is configured to locate in front of the iris of the eye.

77. An implant for insertion into a patient's eye, the implant including an artificial lens and an intraocular device according to claim 52, the artificial lens and the optic member of the intraocular device being positioned in the line of sight such that the patient sees through both.

78. An implant according to claim 77, wherein the optic member covers a central part of the anterior surface of the artificial lens.

79. An implant according to claim 77, wherein the optic member is fused with the central part of the artificial lens.

80. An implant according to claim 77, wherein the intraocular device is configured for location in the ciliary sulcus of the eye, between the lens and the iris.

81. An implant according to claim 77, wherein the intraocular device is configured for location outside the capsular bag, behind the iris, in contact with the ciliary muscle, in a position to be compressed by the contracting ciliary muscle.
